(19) United States
(12) Patent Application Publication
(10) Pub. No.: US 2009/0311738 A1
(43) Pub. Date: Dec. 17, 2009

(54) METHOD AND SYSTEM FOR DETERMINING PROPERTIES OF BIOLOGICAL CELLS

(75) Inventors: Stein Kuiper, Eindhoven (NL); Jacob MARINUS JAN Den Toonder, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/438,178

(22) PCT Filed: Aug. 17, 2007
(86) PCT No.: PCT/IB07/53283

§ 371 Date: Feb. 20, 2009

(30) Foreign Application Priority Data
Aug. 22, 2006 (EP) 06119307.4

(51) Int. Cl. C12Q 1/02; C12M 1/34

(52) U.S. Cl. 435/29; 435/287.1; 435/288.7

(57) ABSTRACT

A system (100) for determining properties of particles is described, wherein elastic properties of particles can be studied. The system (100) typically comprises a microporous structure (110) having a first side and a second side, the microporous structure comprising a plurality of micropores extending from the first side to the second side. Using a means (120) for generating a pressure difference over the microporous structure, particles provided to the first side of the micropores (113) are passed at least partially into the micropores and deformed. A detector (130) is provided for qualitatively and/or quantitatively detecting presence of particles having passed at least partially into the micropores (113), thus allowing to obtain information about the deformation of the particles.

### FIELD OF THE INVENTION

The present invention relates to a system to determine properties of biological particles and to a method to determine properties of biological cells. More in particular, the system relates to determining cytoskeletal properties such as cytoskeletal elasticity and deformability of biological cells.

### BACKGROUND OF THE INVENTION

Detection of malignant cells typically may allow for detection of diseases in an early stage. The latter very often is beneficial, as treatment of diseases in an early stage typically can increase the chance of a successful healing or reduce the chance of a disease developing into a fatal disease. It is well known that cytoskeletal properties of biological cells are correlated with cell function. In particular, it has been shown that the cytoskeletal elasticity changes significantly as a function of cancer progression. By measuring the cell elasticity, therefore, it is in principle possible to detect single malignant cells and precancerous cells—malignantly transformed cells are easier to deform—and this opens the possibility to monitor the progress of cancer from preinvasive to invasive.

US2006/01019836A1 discloses a measurement unit and a method to measure blood particle deformability. The system comprises a blood sample pot and a waste blood pot that communicate by means of a slit channel. The measurement unit further comprises a screen on which diffracted images of the cells are projected, and an image capturing unit to capture the diffracted images. The measuring unit further comprises a differential pressure generator for generating a pressure difference between the blood sample pot and the waste blood pot. Due to the pressure difference, the blood sample will flow through the slit channel from the blood sample pot to the waste blood pot. Due to shear forces working on the blood sample flowing within the slit channel, the blood cells are deformed. Images of deformed blood cells are made using the screen and the image capturing unit. By means of a calculating means, and taking into account the shear forces working on the blood cells, the deformability of the blood cells is calculated.

The existing techniques for measuring the elasticity of cells have a limitation: it requires tedious sample preparation, which limits the number of cells investigated per sample and usually rules out applications in clinical diagnostics. The detection of a small amount of affected cells, having a different deformability as compared with the numerous unaffected cells in the sample is often problematic or difficult.

Also, the presently known methods to measure cytoskeletal properties such as cytoskeletal elasticity and deformability of biological cells requires biological cells to be prepared. As an example, in vivo measurement in human or animal body is not possible, and needs extraction of biological cells.

### SUMMARY OF THE INVENTION

It is an object of the present invention to enable good determination of properties of particles, e.g. biological particles or cells. The above objective is accomplished by a method and device according to the present invention.

The present invention relates to a system for determining properties of particles, the system comprising a microporous structure having a first side and a second side, the microporous structure comprising a plurality of micropores extending from the first side to the second side, a means for generating a pressure difference over the microporous structure for deforming particles provided to the first side of the microporous structure at the micropores and for passing the particles at least partially into the micropores, and a detector for qualitatively and/or quantitatively detecting presence of particles having passed at least partially into the micropores.

Generating a pressure difference over the microporous structure may be generating a pressure that is higher at the first side of the microporous structure than at the second side of the microporous structure. The system as subject of the present invention allows a large number of particles, e.g. biological particles such as e.g. biological cells, to be held by the porous structure but extend to some extent into the pores, even partially through the pores at the second surface, or to pass completely through the pores at a given pressure. The determined properties of the biological particles may enable a physician to make a decision on the presence and the progress of influencing the biological particles, e.g. induced by a cancer, provided the physician takes into account the type of particles which were examined and the type of influencing that is under consideration.

The detector for qualitatively and/or quantitatively detecting presence of particles having passed at least partially into the micropores may be a detector for qualitatively and/or quantitatively detecting presence of particles having passed completely through the micropores.

The microporous structure may have a first major surface at the first side of the microporous structure and a second major surface at the second side of the microporous structure. The means for generating a pressure difference over the microporous structure may be a means for retaining and deforming particles, provided to the first side of the microporous structure, at the micropores. The detector may be a detector for qualitatively and/or quantitatively detecting at a predetermined distance of the first surface the presence of the particles retained by the micropores. The detecting may be detecting in a direction from the first surface to the second surface. It is an advantage of such systems that an efficient system is obtained for detecting properties of a large number of cells simultaneously.

The detector may be adapted for detecting particle material at a predetermined distance from the second surface of the microporous structure, at the second side of the microporous structure. It may be an advantage that the microporous structure material does not contribute to the detected signal.

The detector may be adapted for detecting particle material between the first surface and the second surface, at a predetermined distance from the first surface of the microporous structure. It is an advantage that the shape and elasticity of the particles can be such that particle does not need to be present at the second side of the microporous structure but that it is sufficient to have particle material in the micropores.

The detector may have a limited depth of focus thus allowing to detect particle material present at a substantially predetermined distance of the first surface, either between the first surface and the second surface in the pores, or extending from he second surface. The system has the advantage that differences between particles having a different elasticity can easily be visualized and detected. Depending on the elasticity, for a given pressure difference, the particle will extend over a given length and thus may be present at a distance from the first surface, either still within the pores between the first and the second surface, or extending over a distance from the second surface at a side of the second surface opposite of the first surface. By deriving, e.g. by counting, the number of particles extending from the second surface or extending over a distance between the first and the second surface in the pores, the amount of particles being sufficiently elastic for such extending can be counted for a large number of particles being held by the numerous micropores, thus providing a quantitative result. By measuring the length over which the particles extend from the first surface, the elasticity of the particles can be determined. By measuring the number of particles extending over from the second surface in function of their length extended from the first surface, the elasticity distribution of the sampled particles can be determined.

The detector adapted for detecting particle material at a predetermined distance from the first surface of the microporous structure may be adapted for detecting particle material at a selectable predetermined distance of the first surface of the microporous structure. The system allows visualizing and detecting differences between particles having a different elasticity, and facilitates a more qualitative detection of the particle properties. The system furthermore can be used for detecting particle properties having an elasticity within a large range. The system furthermore allows to accurately distinguish between particles with first particle properties and particles with second particle properties. The detector may comprise a pixelated detector.

The detector may be positioned at the second side of the microporous structure. The system allows easy cleaning of the system, once used. The risk on contamination of components of the system being located at the second side of the porous structure is far less than contamination of components of the system present at the first side. It also enables the porous membrane to contact biological tissue, e.g. during in vivo detection, as nothing of the system is to be present at the first surface of the porous membrane. When the system is placed in cross-flow with the fluid comprising particles of which the properties are to be determined, it allows to refresh the particles, e.g. biological particles present at the first surface by easily back flowing.

The means for generating a pressure difference over the microporous structure may be adapted for generating a plurality of selectable pressure differences over the microporous structure. By making measurements as function of the pressure difference applied over the first and second surface, additional information or properties of the biological particle may be determined. Due to the pressure difference, some more elastic particles may extend from the first to the second surface, and possibly be present at the second side, or may pass through the pores from a particular pressure difference onwards.

The microporous structure may be transparent and the system may be adapted for detecting at the first side a value related to particle size of the particles held at the micropores. This system has the advantage that the size of the biological particles, or the average size, can be measured. The elasticity which may be determined by the presence detection at a determined distance from the first surface, possibly at the second surface, can be adjusted as function of the actual particle diameter or particle size

The detector comprises an illumination source for providing at a second side of the microporous structure an illumination beam substantially parallel with the microporous structure and a detector adapted for detecting light absorption in the illumination beam due to interaction with particle material. It is an advantage of such a detection system that it allows detecting the number of particles extending from the second surface of the microporous structure at given distance from the second surface, or the number of particles passed through the porous structure via the pores, by interpretation of the amount of light absorbed in function of the light absorption coefficient of the particle material.

The detector may comprise an illumination source for providing sidewise incident illumination to particle material at the second side of the microporous structure and a detector being adapted for detecting a shadow obtained by interaction of the illumination and the particle material. This system allows to easily determine the distance of extension from the second surface of the particles present at the second side by measuring the length of the shadow.

The microporous structure may be any of a microporous membrane or a microsieve. The membrane may have substantially flat surfaces, allowing easy detection of presence of particle material at a predetermined distance from the first surface, possibly at the second side of the microporous structure.

The system may further comprise a processing system for calculating the properties of the particles based on the for qualitatively and/or quantitatively detected presence at a predetermined distance from the first surface, possibly at the second surface of particle material of particles held at the micropores or based on the amount of particles passed through the pores for a given pressure difference or in function of the pressure differences used. The system may be a system for determining properties of particles having an average particle diameter, wherein the micropores have a pore diameter between 100 μm and 0.0 μm, such as in the range of 0.1 μm to 100 μm, e.g. between 50 μm and 0.1 μm, more preferred in the range of 1.0 μm to 10 μm

The system may be an endoscopic system. The endoscope can be used to qualitatively and/or quantitatively detecting in vivo the presence at a predetermined distance from the first surface, possibly at the second surface of particle material of the particles held at the micropores. This may avoid extraction of tissue from human of animal body. The endoscope can be used to qualitatively and/or quantitatively detecting in vivo the presence, and the number of, particles passing through the micropores. The endoscopes allow to generate information which can be used by a physician later on make a decision on the presence and the progress of an affection of the biological cells, e.g. an affection due to cancer, provided the physician takes into account the type of particles which were examined and the type of affection that is under consideration.

The present invention also relates to an in-vitro method to determine properties of particles, the method comprising the steps of providing particles to a first side of a microporous structure, deforming the particles at the first side at micropores of the microporous structure by applying a pressure difference across the microporous structure and passing the particles at least partially into the micropores, and qualitatively and/or quantitatively detecting presence of particle material of particles having passed at least partially into the micropores.

The microporous structure may have a first major surface at the first side of the microporous structure and a second major surface at a second side of the microporous structure, and the method may comprise retaining the particles at the first side by applying the pressure difference across the microporous structure, and qualitatively and/or quantitatively detecting a presence of particle material of the particles at a predetermined distance of the first surface in a direction from the first surface to the second surface. The method as subject of the present invention allows a large number of particles to be held by the porous structure. Due to the pressure difference, some more elastic particles may extend from the first to the second surface, extending in the pores between the first surface and the second surface or even extending from the second surface, i.e. be present at the second side, or passing even through the pores. Depending on the elasticity, for a given pressure difference, the particle will extend over a given length from the first surface or even through the pores. By counting the number of particles extending from the first surface over a predetermined distance, possibly by counting the presence of particles at the given micropores along the second surface or even by counting the number of particles passed through the pores, the amount of particles being sufficiently elastic for extension up to a predetermined distance from the first surface or even beyond the second surface even through the pores, can be counted for a large number of particles being held by the numerous micropores. By measuring the length over which the particle extends from the first surface, possibly beyond the second surface, the elasticity of the particle can be determined. By measuring the number of particles extending from the first surface in function of their length extended from the first surface, in particular when the particles extend beyond the second surface, the elasticity distribution of the sampled particles can be determined. By making the above mentioned measurements as function of the pressure difference applied over the first and second surface, additional information or properties of the particles may be determined. The determined properties of the particles may enable a physician to make a decision on the presence and the progress of an affection of the biological cells, e.g. an affection due to cancer, provided the physician takes into account the type of particles which were examined and the type of affection that is under consideration.

The method further may comprise varying the pressure difference over the microporous structure, and qualitatively and/or quantitatively detecting the presence of particle material at a predetermined distance of the first surface as function of the pressure difference. By making the above mentioned measurements as function of the pressure difference applied over the first and second surface, additional information or properties of the particle may be determined.

The method further may comprise measuring the particle size, and using the measured particle size for adjusting the qualitatively and/or quantitatively detected presence at a predetermined distance of the first surface of particle material of particles held at the micropores.

The size of the biological cells, or the average size, may be measured. The elasticity or other properties which may be determined by the presence detection at a predetermined distance of the first surface or even at the second surface, can be adjusted in function of the actual particle diameter or particle size.

The elasticity properties of the particles may be determined based on the qualitatively and/or quantitatively detected presence of particle material. The elasticity typically is one of the properties that change due to affection of biological material, i.e. biological cells. It is a relatively accurate property for a physician to take into account during the making of a diagnosis on the presence and status of affection of biological cells.

The present invention furthermore relates to a method to determine properties of particles, the method comprising providing particles to a first side of a microporous structure, deforming the particles at the first side at micropores of the microporous structure by applying a pressure difference across the microporous structure, and passing the particles at least partially into the micropores, and qualitatively and/or quantitatively detecting presence of particle material of particles having passed at least partially into the micropores.

The microporous structure may have a first major surface at the first side of the microporous structure and a second major surface at a second side of the microporous structure, whereby the method may comprise retaining the particles at the first side at micropores of the microporous structure by applying the pressure difference across the microporous structure and qualitatively and/or quantitatively detecting a presence of particle material of the particles at a predetermined distance of the first surface, in a direction from the first surface to the second surface.

Providing particles to a first side of a microporous structure may comprise endoscopically contacting body tissue with a microporous structure.

The present invention also relates to a processing system for performing a method to determine properties of biological cells, the processing system comprising a detector for qualitatively and/or quantitatively detecting presence of particle material of particles having passed at least partially into the micropores of a microporous structure for particles provided to a first side of the microporous structure and passed at least partially into the micropores of the microporous structure using a pressure difference over the microporous structure, and a system for determining elasticity properties of the particles based on the qualitatively and/or quantitatively determining of the presence of particle material.

The detector for determining presence of particle material may comprise an analyzor for automatically analysing an image of particle material at a predetermined distance from the first surface, possibly at the second side of a microporous structure or particle material passed through the pores of the microporous structure, for a plurality of particles provided to the first side of the microporous structure. The system for determining elasticity properties may comprise means for taking into account the pressure difference and for taking into account mechanical properties of the microporous structure. It may also comprise means for taking into account a measured particle size for determining the elasticity properties.

The present invention furthermore relates to a computer program product for, when executed on a computing means, performing a method to determine properties of particles, the method comprising qualitatively and/or quantitatively detecting presence of particle material of particles having passed at least partially into the micropores of a microporous structure for particles provided to a first side of the microporous structure and passed at least partially into the micropores of the microporous structure using a pressure difference over the microporous structure, and determining elasticity properties of the particles based on the qualitatively and/or quantitatively determining of the presence of particle material.

Determining presence of particle material may comprise automatically analysing an image of particle material at a predetermined distance of the first surface in a direction from the first surface to the second surface, possibly at the second side of a microporous structure for a plurality of particles held at the microporous structure. Determining elasticity properties may comprise taking into account the pressure difference and taking into account mechanical properties of the microporous structure. It may also comprise taking into account a measured particle size for determining the elasticity properties.

The present invention also relates to a machine readable data storage device storing the computer program product as described above and/or the transmission of such computer program products over a local or wide area telecommunications network.

It is an advantage that determination of properties of particles based on measurements done on a large number of particles may be enabled. It is also an advantage that determination of diverging properties of a small number of particles in a large number of particles is facilitated, leading to accurate systems.

Particular and preferred aspects of the invention are set out in the accompanying independent and dependent claims. Features from the dependent claims may be combined with features of the independent claims and with features of other dependent claims as appropriate and not merely as explicitly set out in the claims.

### BRIEF DESCRIPTION OF THE DRAWINGS

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

FIG. 1 is a schematically cross-sectional view of a first system according to embodiments of the present invention.

FIG. 2 is a detailed view of the porous structure as can be used in a system as shown in FIG. 1.

FIG. 3 is an enlarged view of a microsieve as can be used in a system of FIG. 1.

FIG. 4 and FIG. 5 are schematically cross-sectional views of systems according to embodiments of the present invention.

FIG. 6 is a schematic representation of a system being an endoscope, according to embodiments of the present invention.

FIG. 7 shows schematically a processing system for performing a method to determine properties of particles according to embodiments of the present invention.

In the different figures, the same reference signs refer to the same or analogous elements.

### DETAILED DESCRIPTION OF THE EMBODIMENTS

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first and second and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the term over and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

The following terms or definitions are provided solely to aid in the understanding of the invention. These definitions should not be construed to have a scope less than understood by a person of ordinary skill in the art.

The term “optical” and “illumination” in the present application may be visual, infrared or ultraviolet radiation although the invention is not limited thereto. Another part of the electromagnetic spectrum also may be envisaged with this terminology. The term “micropore” is to be understood as a very fine pore with dimensions in micron and submicron range. The term “pore size” is to be understood as the average pore diameter. Typically the average diameter may be between 100 μm and 0.01 μm, such as in the range of 0.1 μm to 100 μm, e.g. between 50 μm and 0.1 μm, more preferred in the range of 1.0 μm to 10 μm. The pores may be circular, however pores may have non-circular, e.g. rectangular or oval shapes. In case on non-circular pores, “average diameter” of a given pore is to be understood as the diameter of an imaginary circle having the same surface area as the given pore.

The term “pore” is to be understood as an open area extending from one side of the structure to the other side of the structure, i.e. forming a channel. The pore preferably has substantially constant cross sectional dimensions, i.e. a substantially constant diameter in case of circular cross sections, or substantially constant length and width in cross-section in case the pores have a slit-like cross section.

The term “a distance ‘x’ from the first surface of the microporous structure” is thereafter to be understood as a distance ‘x’ measured in a direction from the first surface towards the second surface, thus extending towards the second surface, away from the first surface. The term “a distance ‘y’ from the second surface of the microporous structure” is thereafter to be understood as a distance ‘y’ measured in a direction from the first surface towards the second surface, thus extending away from both the first and the second surface.

The term “particle size’ of a given particle is to be understood as the diameter of an imaginary circle having the same volume of the given particle. The particles under study may e.g. be biological particles such as biological cells including but not limited to prokaryotic cells such as bacterial cells or fungal cells, eukaryotic cells such as mammalian cells, or particle derivates such as spheroblasts. The biological particles may also be small tissue clusters or cell clusters. The particles may also be inanimate particles such as polymer beads, e.g. latex or polystyrene beads, artificial cells such as e.g. liposomes, micelles, polymerosomes, microcapsules, etc. Eukaryotic cells may include any cells that are used in diagnostics, e.g. cells from suspected cancerous tissue, epithelial cells, lymphocytes, macrophages, fibroblasts, PC12 cells, keratinocytes and melanoma cells. The particles preferably are spherical particles, although the invention is not limited thereto. They also may be non-spherical particles such as e.g. muscle and neuronal cells, fibroblasts, or other non-spherical cells such as e.g. rod-shaped bacteria. The pores may e.g. also be rectangular or oval.

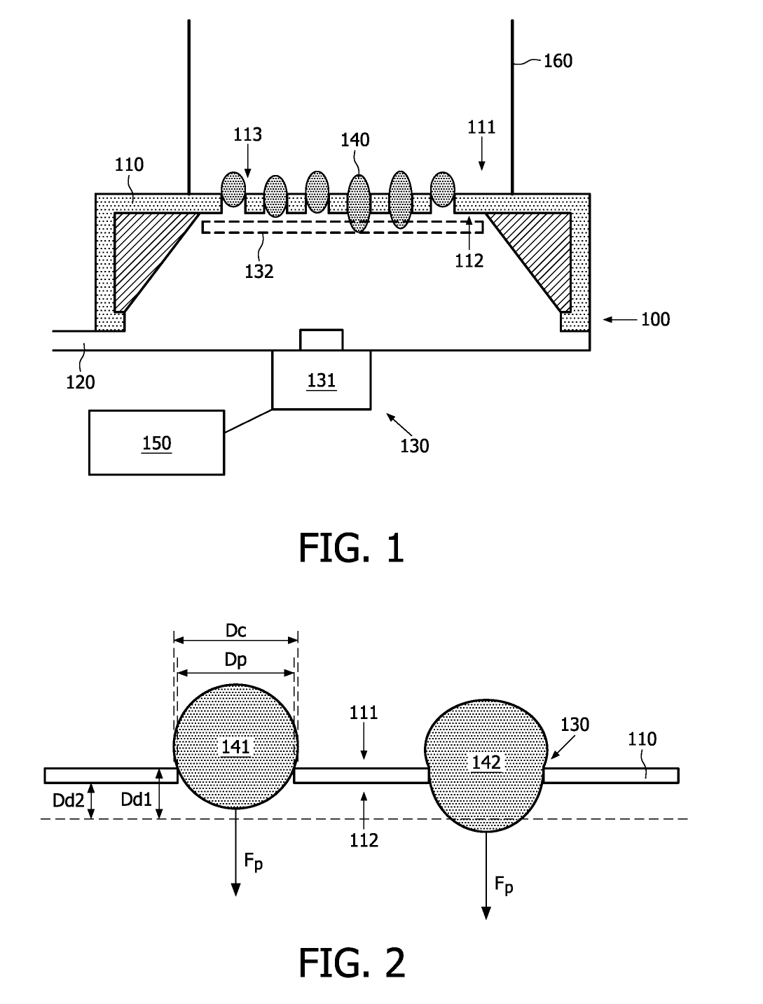

In a first embodiment of the first aspect of the present invention, a system for determining properties of particles, e.g. biological particles, as subject of the present invention is provided. An exemplary system is schematically shown in cross-section in FIG. 1. The system 100 comprises a microporous structure 110 having a first side formed by a first major surface 111 and a second side formed by a second major surface 112 of the structure, the structure also comprising a plurality of micropores 113 extending from the first side to the second side. The second side typically may be opposite the first side with respect to the microporous structure 110. The system 100 furthermore comprises a means 120, 160 for generating a fluid pressure difference across the microporous system, i.e. a pressure higher at the first side than at the second side, for deforming particles 140 provided to the micropores and for passing the particles at least partially into the micropores and in this embodiment for retaining or holding particles 140 provided to the first side in the micropores. This means is also referred to as pressure difference generator. This means to generate fluid pressure over the microporous system may be a means 120 to create a sub pressure at the second side of the microporous structure 110, a means 160 to provide an overpressure to the first side of the microporous structure 110, or a combination of both. The system 100 may also comprise a detector 130 comprising a detector 131 for qualitatively and/or quantitatively detecting presence of particle material of the particles at a predetermined distance from the first surface 111, i.e. extending in the micropores up to a distance between the first surface 111 and the second surface 112 in the micropores, or even beyond the second surface 112 at the second side of the microporous structure 110, which particles are held at the micropores. The detector 131 may also be useful to detect other qualitative or quantitative characteristics or properties of the particle material held at the micropores which are deformed and at least partially passed into the micropores. The detector 130 may be suitable for measuring deformation while the cell membrane is somewhere between the first and second surface. Detecting a presence of particle material at the second side may also include detecting presence of particle material at a predetermined distance from the second major surface 112 at the second side of the microporous structure 110. Measuring from the first surface (111), the distance over which the particle material bulges from the first surface towards the second surface, and possibly beyond the second surface, is typically between 0% and 200% of the pore size, i.e. the average pores diameter.

In use, particles are hold or ‘trapped’ on the pores 113 of the microporous structure 110 at the first major surface 111 of the structure, such as a microporous membrane, by means of a hydrostatic pressure difference over the structure, e.g. a membrane. As shown in more detail in FIG. 2, if the smallest diameter Dc is larger than the average pore diameter Dp, the particle 141, 142 will be pushed into the pore 113 and the particle wall will deform. The system is especially useful to study particles with an average diameter such that the ratio particle diameter/pore diameter is within the range 1.01 to 10, preferably 1.1 to 2. The deformation is dependent on the pressure difference over the microporous structure 110, the ratio of particle size and pore size, and the wall elasticity of the particle 141, 142. Since cells may vary strongly in size, the pore diameter may be kept significantly smaller than the average particle size. From the rear (exit) side of the microporous structure, i.e. towards the second major surface 112, the presence of a particle 141 or 142 can be measured at this second surface 112, at a given distance from the second surface 112 or at a given distance from the first surface extending between the first and the second surface. In the present application, the rear side of the microporous structure 110 is that side of the microporous structure 110 to which particles to be studied are not pressed by the pressure differential. From detection of the presence or visibility of a particle at a distance from the first side, preferably when extending beyond the second side, at a given pressure, the deformation and hence the elasticity of the particle can be determined. It is an advantage that a microporous structure with many pores allows for a simultaneous trapping and measuring of many particles. A particle to be identified 142 having an altered elasticity, e.g. mutated or affected cells, will bulge further or less far in and through the pores 113 compared to particles having “normal” elasticity, making them easily detectable. By qualitatively detecting the presence of at least one particle at a distance Dd1 from the first surface, and/or Dd2 from the second surface 112, the presence of particles 142 with altered elasticity can be detected within the numerous particles 141 with ‘normal’ elasticity. In order to determine the ‘normal’ or reference elasticity of particles, a calibration measurement on only healthy particles may be executed. Knowing the number of pores 113 per surface unit of the porous structure, and taking into account a filling degree of the pores, e.g. a 100% filling of the pores (i.e. each pore is provided with one cell), the amount of particles having increased elasticity as compared with the total amount of particles or the amount of particles with normal elasticity can be determined by quantitatively counting the number of particles present at distance Dd1 and/or Dd2.

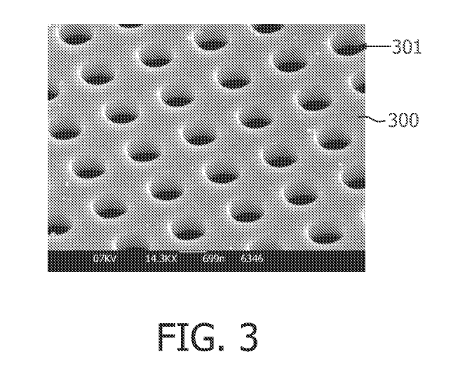

For an easy detection it is preferred that the microporous structure 110 is thin and flat. A typical thickness of the microporous structure 110 may be such that it is substantially smaller than the average diameter of the particles, e.g. biological cells, to be studied. The thickness may e.g. be in the range 1.0 times the average pore diameter or smaller, e.g. 0.1 times the average pore diameter or smaller. A thickness of 0.1 μm to 5.0 μm is preferred. A suitable example of a microporous structure 110, the invention not being limited thereto, are microsieves. These are thin membranes with uniform pores 301, e.g. made by etching. A lithographic process may be performed for generating a pattern of pores in the membrane. Other types of microporous structures 110 also may be used, such as e.g. track-etched membrane may be used. An example of such a microsieve 300 is given in FIG. 2 and FIG. 3. As an example, the pore size of the pores 301 in FIG. 3 is 0.7 μm, but the design can easily be adjusted to a size that matches the particles that must be analysed. Typically the microporous structure 110 may be made from any suitable material, for example a ceramic material, a semiconductor material such as e.g. silicon nitride or silicon oxide, or a polymer material such as e.g. polyamide or polycarbonate, or silicon carbide, aluminium oxide, metal (e.g. copper, nickel, gold, silver, aluminium, titanium). In general as shown in FIG. 1, FIG. 2 and FIG. 3, it is preferred that for determining properties of biological particles having a given average particle diameter Dc, the micropores 113, 301 have a pore diameter Dp of 0.1 μm to 50 μm. The number of micropores present in the membrane may be large. The number of micropores may e.g. be such that a porosity of more than 1%, preferably more than 10%, still more preferably of more than 20% is obtained. In case the ratio of pore size and particle size is small, i.e. the particles are significantly lager than the average pore size, lower porosities are preferred. This in order to avoid particles covering more than one pore of the membrane. The larger the number of micropores, the more sites are provided for detecting particle properties.

With regard to the pressure to be applied, suppose the pore diameter is 10 μm. The required pressure difference for a resulting force Fₚ of 1 nN is equal to the ratio of force and pore area A:

$$Δ p = \frac{{F}_{p}}{A} = \frac{{10}^{- 9}}{{π ( 5 \cdot {10}^{- 6} )}^{2}} = 13 Pa$$

13 Pa is a very low pressure difference. The microporous structures such as membrane or microsieves usually can resist pressure differences above 1 bar (=10⁵ Pa). In other words: large deformations can be obtained as larger pressure differences are allowed by microporous structures as described above, which is advantageous for detection. Especially when bulging beyond the second surface is measured, i.e. the length of extension Dd2 from the second surface in function of pressure difference, a measure for the particle wall elasticity can be determined. The membrane or microsieves should be designed such that the membrane or sieve does not deflect to a too large extent during pressurization. The deflection of the membrane or microsieves should be much smaller than Dd2. Typically, this may mean that the sieve should be thick enough to prevent too large deflections, or that the distance between the support bars of the sieve is small enough to make the deflection small.

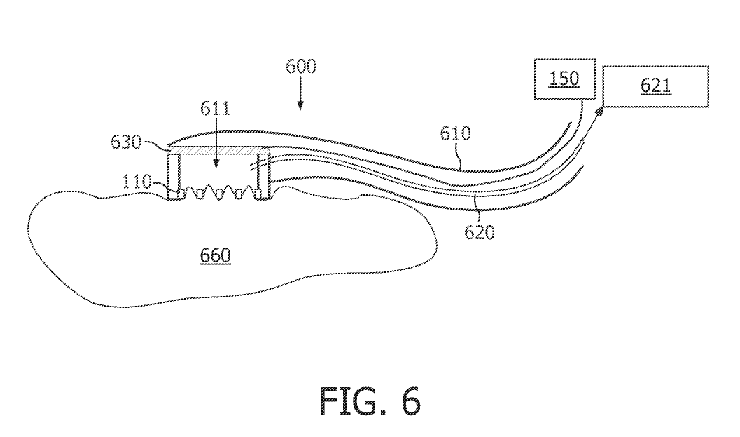

The particles can be detected from the second major or rear side of the microporous structure with various techniques as shown in FIG. 1, FIG. 5 and FIG. 6 by way of example, the invention not being limited thereto. Optical techniques or visual detection with a microscope is possible. As shown in FIG. 1, if the detector 131 being used is a microscope and it has a small depth of focus e.g. a confocal microscope, as indicated with the focus field 132, the presence of particle material in this field can be visualised and possibly be detected and counted by means of image analysis techniques. If the detector 131 is a microscope and has a small depth of focus as indicated with the field 132, and the distance between the first surface 111 of the microporous structure 110 and the microscope 131 is adjustable, such as adjustable over the distance between the second surface 112 of the microporous structure 110 and the microscope 131, a variation in the relative distance between the imaged field and the microporous structure position in the direction substantially perpendicular to the membrane, i.e. substantially perpendicular to an average plane through the membrane, typically may result in different, or more particularly more or less particles being imaged. The latter may show differences between particles having a modified elasticity, e.g. affected particles and other normal cells. In other words, it is also possible to obtain information about the shape of the particles. For example by systematically moving the focus plane of the microscope towards 112 (i.e. varying Dd) and carrying out a measurement, the shape of the particles may be imaged. The latter may e.g. be based on 3D imaging using confocal microscopy. Another exemplary technique that can be used is interferometry.

Sometimes it is difficult to see particles with visible light. For example, detection can be done with UV light (high absorption) or optical detection can be improved by staining the particles, e.g. cells to improve the visibility of the particle material of the particles.

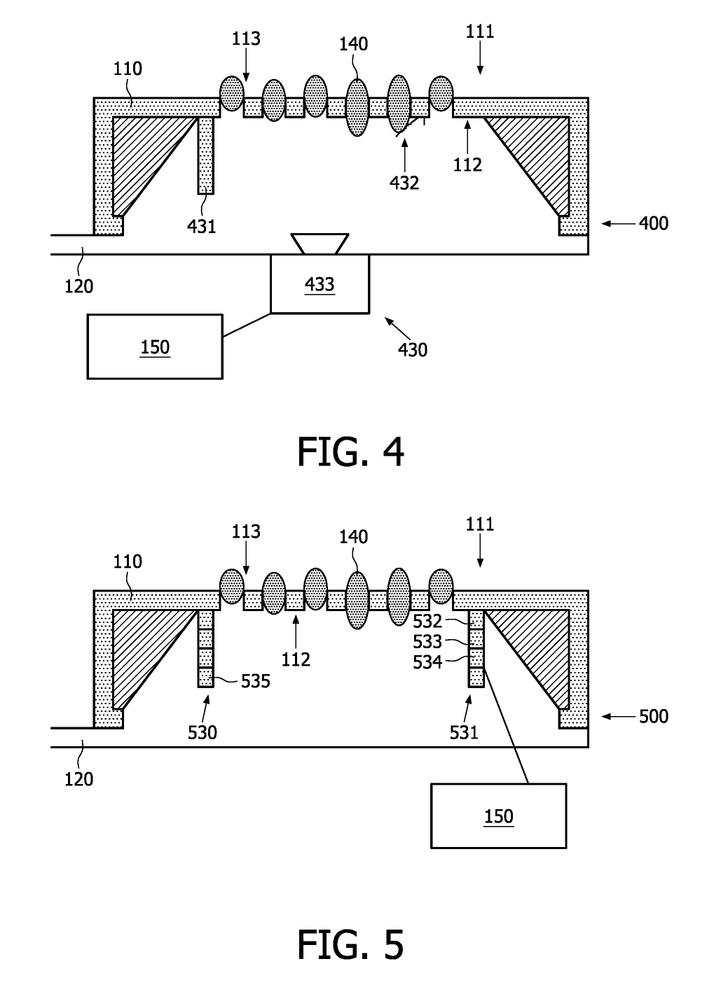

An alternative system 400 for detecting properties of particles is shown in FIG. 4. Identical numbers refer to identical features as in FIG. 1. The detecting means 430 comprises an illumination source 431 which provides glancing or sidewise incident light to the second major surface 112 of the porous structure 110. The illumination source 431 illuminates the particles from the side, and the length 432 of the shadows are a measure for how far the particle extends or bulges through the pore 113. The length of the shadow is measured from images captured by an image-capturing unit 433. The pressure difference determines the particle deformation. The length of the shadows can be calculated to height of extension for each particle extending from the second surface 112. The elasticity of the particles can be determined therefrom.

Yet another alternative system 500 is shown in FIG. 5. Identical numbers refer to identical features as in FIG. 1. The detecting means 530 comprises a detector 531 for measuring light intensity at the second side underneath the microporous structure 110 and an illumination source 535 diametrically opposite but also underneath the microporous structure 110 for illuminating an area underneath the microporous structure 110. Depending on the number of particles extending sufficiently far from the second side of the microporous structure 110, the illumination beam will be more or less attenuated, i.e. absorbed. The light intensity thus may be a measure for the number of particles extending far through the microporous structure 110, and thus may provide information on the concentration of affected particles. In other words, detecting the number of particles extending from the second surface of the microporous structure at given distance from the second surface can be done by interpretation of the amount of light absorbed. The latter also is determined by the absorption coefficient of the particle material. The detector 531 also may comprise a plurality of detector elements 532, 533, 534, as indicated in FIG. 5, allowing measurement of light intensities for illumination beams passing parallel with the microporous structure 110 at different distances from the second surface of the microporous structure 110. The latter may provide information about the difference in elasticity of different particles present in the sample.

It is to be understood that a processing system 150 may be provided to determine properties of particles by calculating them from the obtained information on presence of particles at given distance from the second surface of the porous structure 110, e.g. from the optical signals detected. Such a processing system 150 may be e.g. a microprocessor, and/or a memory component for storing the obtained and/or processed information. Furthermore typical input/output means may be present. The processing system 150 may be controlled using appropriate software or dedicated hardware processing means for executing the calculation steps. The processing system thus may be implemented in any suitable manner, e.g. dedicated hardware or a suitably programmed computer, microcontroller or embedded processor such as a microprocessor, programmable gate array such as a PAL, PLA or FPGA, or similar. The results may be displayed on any suitable output means such as a visual display unit, plotter, printer, etc. The processing system 150 may also have a connection to a local area or wide area network for transmission of the results to a remote location. The processing system 150 may be adapted for performing image recognition techniques, allowing automated recognition of the presence of a particle in an image. In order to process the optical results obtained to particle properties, e.g. deformation properties of particles, predetermined algorithms, neural networks or other suitable means may be used. The latter may be performed in an automated and/or automatic way.

The system 500 of FIG. 5 may alternatively be used as a system wherein the detector 530 for qualitatively and/or quantitatively detecting presence of particles having passed completely through the micropores. The detector 130 may qualitatively and/or quantitatively detect presence of particles passed through the micropores when leaving the second surface of the miroporous structure. The latter may be performed using similar techniques as when the detector is used to detect the presence of particle material at a particular distance from the second surface, for particles substantially retained at one side of the microsieve. By interpretation of the images or data obtained by the detector 130, the amount or number of particles passing through the microporous structure may be counted or deduced. These methods are based on the fact that at very high pressure difference, the most elastic cells will be pushed through the micropores Possibly the number of particles as function of pressure differences over the microporous structure may be generated, which may enable to provide information about the difference in elasticity of different particles present in the sample. Alternatively or combined therewith, the threshold pressure may be determined for particles passing through the micropores, which may give an indication about the elasticity of particles present in the sample. In other words, detecting at which pressure particles start to pass completely through the pore may be a way to detect an affection.

In a second embodiment according to the first aspect of the present invention, a system for detecting particle properties as described above is provided, wherein furthermore the system is adapted for taking into account the particle size distribution in the sample. Whereas the particle size is not very relevant if the particle is much larger than the pore, the latter may become more relevant if the particle size becomes of the same order as the pore size. Typically, small particles will bulge further in, through and/or out of the pores than large particles, due to the smaller radius of curvature of their walls. It is, however, possible to correct for the particle size. Using e.g. a transparent microporous structure 110 allows measurement of the particle size by looking through the microporous structure 110, e.g. by using the microscope 131 having small depth of field and determining the particle size from the first side of the microporous structure 110. Typically, transparent microporous structures 110 may be made from e.g. silicon nitride or silicon oxide. Alternatively, another system, e.g. optical system, may be provided for determining the particle size distribution. The particle size distribution information obtained typically may be provided to the processing system 150, thus allowing to adjust the determined properties of the particles using the measured individual particle size or the average particle size made by averaging the sizes of different particles measured. In an alternative embodiment standard distributions of the particle size based on e.g. previous measurements, literature values or look up tables may be provided to the processing system 150.

In a third embodiment according to the first aspect, the present invention provides a system for detecting particle properties as described in any of the first or second embodiments, wherein furthermore the system is adapted for providing quantitative information about different variations in properties of the particles. In the third embodiment, the system is adapted for performing measurements at different pressures, allowing to obtain values relating to a qualitative or quantitative variation of the properties of the particles under study. The latter may be made possible by providing a means for generating a plurality of different pressures over the porous structure 110, i.e. a means for varying the pressure over the porous structure 110 for instance in a sequential manner. The latter may e.g. be obtained by using a controllable pump. By consecutively detecting the presence of particle material at a predetermined distance of the second surface of the microporous structure, information about the degree of elasticity change may be obtained. The latter also may be obtained by a applying a given pressure and detecting the presence of particle material at different distances form the first or second surface of the microporous structure. The latter may be performed by controlling the detection unit for detecting such presence at different distances from the microporous structure. In either case, an idea of the range and distribution of elasticity of the particles is obtained. This information can be used to determine the number of particles having an elasticity in a given range. The latter may provide information about the degree of malignantly transformation of the particles under study and the distribution thereof.

The embodiments of a system according to the present invention may further comprise a means for reversing the pressure difference over the microporous structure, for cleaning membrane. By providing a reversed pressure over the microporous structure, the particles present, and possibly hold, at the first surface of the microporous structure are removed. This means for reversing the pressure difference may be part of the means for generating pressure difference over the microporous structure, or may be a separate means.

The embodiments of a system according to the first aspect may further comprise a cleaning channel on the second side for enabling the cleaning of the system by means of crossflow cleaning, i.e. by using a liquid flow.

Optionally, the microporous structure is a disposable porous structure, such as a disposable sieve. This is especially preferred in case the system is for in vivo use, as the disposability increases the patient hygiene, reduces the risk on infection due to incomplete disinfection and sterilisation.

When the system according to the first aspect of the present invention is to be used for analysing particles, such as cells, in liquids, the detecting means is preferably positioned above the liquid to be sampled to avoid the detector to moisten and possibly be contaminated by the liquid. In a second aspect, the present invention relates to a method for detecting properties of particles, e.g. biological particles. The method typically may allow to derive cytoskeletal properties, such as e.g. information about the elasticity of particles, e.g. biological particles. Systems for detection as described in embodiments according to the first aspect are especially suitable for performing a method as described in the present embodiment. The method typically comprises providing particles, e.g. biological cells, to a first side, e.g. at a first major surface, of a microporous structure. It furthermore comprises generating a pressure difference over the microporous structure, which may be provided by generating an overpressure at a first side of the microporous structure where the particles are provided or by generating a low or sub pressure at the second side, opposite to the first side of the microporous structure. By generating such a pressure difference, the particles are deformed and at least partially passed into the pores of the microporous structure. Optionally, the particles are held in the micropores at the first surface of the microporous structure. The method furthermore comprises qualitatively and/or quantitatively detecting, preferably from the second side of the microporous structure, presence of particle material of the particles, e.g. biological particles, having passed at least partially into the micropores. This may be a detection of particles having passed through the microporous structure, or detecting presence of particle material held at the micropores, being present on a predetermined distance from the first surface of the miroporous structure, i.e. particles extending in the pores up to a distance between the first and second surface of the microporous structure, or particles present at the second side of the porous structure, on a predetermined distance from the second surface. Detecting presence at the second side of the microporous structure near the second surface of particle material of the particles typically may be detecting the presence of particle material at a predetermined distance of the microporous structure, at the second surface of the microporous structure. Detecting presence of particle material of the particles between the first and second surface of the porous structure typically may be detecting the presence of particle material at a predetermined distance of the first surface of the microporous structure. Optionally, the pressure difference across the first and second side may be varied and the detection may be performed for different pressure values. In other words, a qualitative and/or quantitative detection of presence of particle material as function of the applied pressure difference can be performed. Using the information on the detected particle material, optionally detected at different distances from the first surface and/or at different distances from the second surface of the porous structure and/or when different pressure differences are used, properties can be determined from the particles provided to the first surface of the porous structure. Such properties, e.g. the elasticity of the particle or particle wall, can be determined by transforming the information on presences using appropriate algorithms. This can be done in a step of processing the detected information.

Optional additional steps of measuring the particle size and using the measured particle size for adjusting the properties of the particles based on the for qualitatively and/or quantitatively detected presence at the second surface of particle material of particles held at the micropores are applied. This can provide a more accurate determination of the particle property.

The method may be an in-vitro method. Alternatively, the method also may be performed in-vivo, e.g. using an endoscopic system as described in the third aspect of the present invention.

In a third aspect, the present invention relates to a system as described in the first aspect, whereby the system is endoscope 600 as shown schematically in FIG. 6. The endoscopic system thus typically allows determining properties of particles, e.g. biological particles, and comprises the same features and advantages as a system described in the first aspect of the present invention. Again identical references refer to identical features. The microporous structure is provided at the end portion of the endoscopic system and is adapted for bringing it with its first side in contact with particles, e.g. biological tissue. The detector 630 of the present embodiment is provided in the endoscopic system and allows to detect deformation of particles by detecting particle material at a second side of the microporous structure. Typically the second side of the microporous structure may be positioned in a chamber 611 that can be brought at a pressure lower than the environmental pressure, in this way generating a pressure difference over the microporous structure for holding particles to micropores in the microporous structure at the first side of the microporous structure. The endoscope furthermore typically may comprise a tubular wall 610 in which means may be provided for coupling the chamber 611 to a pump 621, external to the endoscope, by means of a tube 620 and to couple the detecting means 630 to a processing system 150, external to the endoscope. The endoscope can be inserted in the human or animal body, to bring the porous structure into contact with the particles, e.g. tissue 660, to be measured. A pressure difference is generated, e.g. using pump 621 creating a sub pressure in the chamber 611, thus attracting the tissue 660 particles and holding them at the micropores. The sub pressure deforms the particles contacting the first surface of the porous structure and using the detector 630 and the processing system 150, the number of particles having an increased or decreased elasticity as compared to normal tissue can be determined. As set out above, by varying the pressure difference and by detecting the presence of particle material at different heights from the second surface, also the elasticity of the particles measured can be determined. In a particular case, i.e. in the case of a cancer, the affected particles deform more than in case of healthy cells. Hence by using the endoscope 600 in vivo, the particle properties can be determined without the need to take away tissue. Based on the information regarding the particle properties, a physician may make a decision on the presence and the progress of an affection of the biological cells, e.g. affection due to cancer, provided the physician takes into account the type of particles which were examined and the type of affection that is under consideration. It is understood that the systems as subject of the present invention may as well be used to determine properties of particles ex-vivo, e.g. by suspending sample particles in a physiological salt solution and contacting this with the porous structure. If a batch of particles has been examined, a simple cross flow system can remove the particles and supply new cells.

It is understood that the endoscope 600 may comprises a detector detecting presence of particle material at a predetermined distance from the first surface, the particle material being present between the first and second surface of the microporous structure.

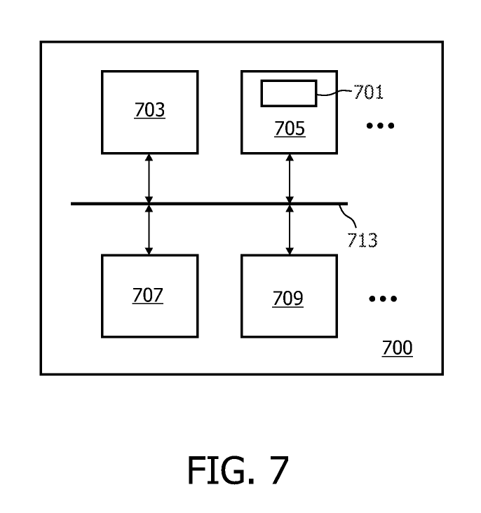

The above-described method embodiments of the present invention may be implemented in a processing system 700 such as shown in FIG. 7. Such a system may e.g. be a system for performing a method to determine properties of biological cells. Such a processing system may comprise a means for determining qualitatively and/or quantitatively the presence of particle material at a distance from the first surface of the microporous structure, and/or at a distance from the second side of a microporous structure (110) for particles provided to a first side of a microporous structure (110) and optionally held at micropores of the microporous structure (110) using a pressure difference over the microporous structure (110), and a means for determining elasticity properties of the particles based on the qualitatively and/or quantitatively determining of the presence of particle material. The means for determining presence of particle material may comprise means for automatically analysing an image of particle material at the second side of a microporous structure for a plurality of particles optionally held at the microporous structure. Any system for automated feature recognition therefore may be used. The means for determining elasticity properties may comprise means for taking into account the pressure difference and for taking into account mechanical properties of the microporous structrure. Typically such parameters are input values for the means for determining elasticity properties. Typically the determining elasticity properties may make use of a look-up table (LUT), a predetermined algorithm or a neural network. It may also comprise means for taking into account a measured particle size for determining the elasticity properties. FIG. 7 shows one configuration of such a processing system 700 that includes at least one programmable processor 703 coupled to a memory subsystem 705 that includes at least one form of memory, e.g., RAM, ROM, and so forth. It is to be noted that the processor 703 or processors may be a general purpose, or a special purpose processor, and may be for inclusion in a device, e.g., a chip that has other components that perform other functions. Thus, one or more aspects of the present invention can be implemented in digital electronic circuitry, or in computer hardware, firmware, software, or in combinations of them. The processing system may include a storage subsystem 707 that has at least one disk drive and/or CD-ROM drive and/or DVD drive. In some implementations, a display system, a keyboard, and a pointing device may be included as part of a user interface subsystem 709 to provide for a user to manually input information. Ports for inputting and outputting data also may be included. More elements such as network connections, interfaces to various devices, and so forth, may be included, but are not illustrated in FIG. 7. The various elements of the processing system 700 may be coupled in various ways, including via a bus subsystem 713 shown in FIG. 7 for simplicity as a single bus, but will be understood to those in the art to include a system of at least one bus. The memory of the memory subsystem 705 may at some time hold part or all (in either case shown as 711) of a set of instructions that when executed on the processing system 700 implement the steps of the method embodiments described herein. Thus, while a processing system 700 such as shown in FIG. 7 is prior art, a system that includes the instructions to implement aspects of the methods for detecting properties of particles is no prior art and therefore FIG. 7 is not labelled as prior art.

The present invention also includes a computer program product which provides the functionality of any of the methods according to the present invention when executed on a computing device. Such computer program product may e.g. be for performing a method to determine properties of biological cells. It may be adapted for determining qualitatively and/or quantitatively the presence of particle material at a second side of a microporous structure (110) for particles provided to a first side of a microporous structure (110) and held at micropores of the microporous structure (110) using a pressure difference over the microporous structure (110), and for determining elasticity properties of the particles based on the qualitatively and/or quantitatively determining of the presence of particle material. The determining presence of particle material may comprise automatically analysing an image of particle material at the second side of a microporous structure for a plurality of particles held at the microporous structure. Any system for automated feature recognition therefore may be used. The determining elasticity properties may comprise taking into account the pressure difference and taking into account mechanical properties of the microporous structure. Typically such parameters are input values for determining elasticity properties. Typically the determining elasticity properties may make use of a look-up table (LUT), a predetermined algorithm or a neural network. It may also comprise taking into account a measured particle size for determining the elasticity properties. Such computer program product can be tangibly embodied in a carrier medium carrying machine-readable code for execution by a programmable processor. The present invention thus relates to a carrier medium carrying a computer program product that, when executed on computing means, provides instructions for executing any of the methods as described above. The term “carrier medium” refers to any medium that participates in providing instructions to a processor for execution. Such a medium may take many forms, including but not limited to, non-volatile media, and transmission media. Non volatile media includes, for example, optical or magnetic disks, such as a storage device which is part of mass storage. Common forms of computer readable media include, a CD-ROM, a DVD, a flexible disk or floppy disk, a tape, a memory chip or cartridge or any other medium from which a computer can read. Various forms of computer readable media may be involved in carrying one or more sequences of one or more instructions to a processor for execution. The computer program product can also be transmitted via a carrier wave in a network, such as a LAN, a WAN or the Internet. Transmission media can take the form of acoustic or light waves, such as those generated during radio wave and infrared data communications. Transmission media include coaxial cables, copper wire and fibre optics, including the wires that comprise a bus within a computer.

It is to be understood that although preferred embodiments, specific constructions and configurations, as well as materials, have been discussed herein for devices according to the present invention, various changes or modifications in form and detail may be made without departing from the scope and spirit of this invention.

1. A system (100, 400, 500, 600) for determining properties of particles, the system (100) comprising
a microporous structure (110, 300) having a first side and a second side, the microporous structure (110, 300) comprising a plurality of micropores (113, 301) extending from the first side to the second side,
a means (120, 160) for generating a pressure difference over the microporous structure (110) for deforming particles provided to the first side of the microporous structure (110) at the micropores (113) and for passing the particles at least partially into the micropores, and
a detector (130, 430, 530, 630) for qualitatively and/or quantitatively detecting presence of particles having passed at least partially into the micropores.

2. A system (100, 400, 500, 600) according to claim 1, the microporous structure (110, 300) having a first major surface (111) at said first side of said microporous structure (110) and a second major surface (112) at said second side of said microporous structure (110), wherein
the means (120, 160) for generating a pressure difference over the microporous structure (110) is a means for retaining and deforming particles provided to the first side of the microporous structure (110) at the micropores (113), and
the detector (130, 430, 530, 630) is a detector for qualitatively and/or quantitatively detecting at a predetermined distance of the first surface (111) in a direction from the first surface (111) to the second surface (112), the presence of the particles retained by the micropores (113).

3. A system (100, 400, 500, 600) according to claim 2, wherein the detector (130, 430, 530, 630) is adapted for detecting particle material at a predetermined distance from the second surface (112) of the microporous structure (110), at the second side of the microporous structure (110).

4. A system (100) according to claim 2, wherein the detector (130) is adapted for detecting particle material between the first surface (111) and the second surface (112), at a predetermined distance from the first surface (111) of the microporous structure (110).

5. A system (100, 400, 500, 600) according to claim 2, wherein the detector (130, 430, 530, 630) adapted for detecting particle material at a predetermined distance from the first surface (111) of the microporous structure (110) is adapted for detecting particle material at a selectable predetermined distance of the first surface (111) of the microporous structure (110).

6. A system (500) according to claim 1, wherein a detector (530) for qualitatively and/or quantitatively detecting presence of particles having passed at least partially into the micropores is a detector for qualitatively and/or quantitatively detecting presence of particles having passed completely through the micropores.

7. A system (100, 400, 500, 600) according to claim 1, wherein the detector (130, 430, 530, 630) is positioned at said second side of the microporous structure (110).

8. A system (100, 400, 500, 630) according to claim 1, wherein said means (120, 160) for generating a pressure difference over the microporous structure (110) is adapted for generating a plurality of selectable pressure differences over the microporous structure (110).

9. A system (100) according to claim 3, wherein the microporous structure (110) is transparent and said system (100) is adapted for detecting at the first side a value related to particle size of said particles held at the micropores (113).

10. A system (500) according to claim 1, wherein the detector (530) comprises an illumination source (535) for providing at a second side of said microporous structure (110) an illumination beam substantially parallel with said microporous structure (110) and a detector (531) adapted for detecting light absorption in said illumination beam due to interaction with particle material.

11. A system (400) according to claim 4, wherein the detector (430) comprises an illumination source (431) for providing sidewise incident illumination to particle material at the second side of the microporous structure (110) and a detector (433) being adapted for detecting a shadow obtained by interaction of the illumination and the particle material.

12. A system (100, 400, 500, 600) according to claim 1, wherein the microporous structure (110, 300) is any of a microporous membrane or a microsieve.

13. A system (600) as in claim 1, the system (600) being an endoscopic system.

14. An in-vitro method to determine properties of particles, the method comprising the steps of
providing particles to a first side of a microporous structure (110),
deforming said particles at said first side at micropores of said microporous structure (110) by applying a pressure difference across said microporous structure (110), and passing the particles at least partially into the micropores, and
qualitatively and/or quantitatively detecting presence of particle material of particles having passed at least partially into the micropores.

15. An in-vitro method to determine properties of particles according to claim 14, the microporous structure (110) having a first major surface (111) at said first side of said microporous structure (110) and a second major surface (112) at a second side of said microporous structure (110), the method comprising
retaining said particles at said first side at micropores of said microporous structure (110) by applying the pressure difference across said microporous structure (110), and
qualitatively and/or quantitatively detecting a presence of particle material of said particles at a predetermined distance of the first surface (111) in a direction from the first surface (111) to the second surface (112).

16. An in-vitro method according to claim 15, the method further comprising
varying the pressure difference over the microporous structure (110), and
qualitatively and/or quantitatively detecting the presence of particle material at a predetermined distance of the first surface (111) as function of the pressure difference.

17. An in-vitro method according to claim 15, the method further comprising
measuring the particle size, and
using the measured particle size for adjusting the qualitatively and/or quantitatively detected presence at a predetermined distance of the first surface (111) of particle material of particles held at the micropores.

18. An in-vitro method according to claim 15, wherein the elasticity properties of the particles are determined based on said qualitatively and/or quantitatively detected presence of particle material.

19. A method to determine properties of particles, the method comprising the steps of
providing particles to a first side of a microporous structure (110), the microporous structure (110) having a first major surface (111) at said first side of said microporous structure (110) and a second major surface (112) at a second side of said microporous structure (110),
deforming said particles at said first side at micropores of said microporous structure (110) by applying a pressure difference across said microporous structure (110), and passing the particles at least partially into the micropores, and
qualitatively and/or quantitatively detecting presence of particle material of particles having passed at least partially into the micropores.

20. A method to determine properties of particles according to claim 19, the method comprising
retaining said particles at said first side at micropores of said microporous structure (110) by applying the pressure difference across said microporous structure (110),
qualitatively and/or quantitatively detecting a presence of particle material of said particles at a predetermined distance of the first surface (111) in a direction from the first surface (111) to the second surface (112).

21. A method according to claim 19, wherein the providing particles to a first side of a microporous structure (110) comprises endoscopically contacting body tissue with a microporous structure (110).

22. A processing system for performing a method to determine properties of biological cells, the processing system comprising
a detector for qualitatively and/or quantitatively detecting presence of particle material of particles having passed at least partially into the micropores of a microporous structure for particles provided to a first side of the microporous structure (110) and passed at least partially into the micropores of the microporous structure (110) using a pressure difference over the microporous structure (110), and
a system for determining elasticity properties of said particles based on said qualitatively and/or quantitatively determining of the presence of particle material.

23. A computer program product for, when executed on a computing means, performing a method to determine properties of particles, the method comprising
qualitatively and/or quantitatively detecting presence of particle material of particles having passed at least partially into the micropores of a microporous structure for particles provided to a first side of the microporous structure (110) and passed at least partially into the micropores of the microporous structure (110) using a pressure difference over the microporous structure (110), and
determining elasticity properties of said particles based on said qualitatively and/or quantitatively determining of the presence of particle material.

24. A machine readable data storage device storing the computer program product of claim 23.

25. Transmission of the computer program products of claim 23 over a local or wide area telecommunications network.
